(19) United States
(12) Patent Application Publication
(10) Pub. No.: US 2024/0374372 A1
(43) Pub. Date: Nov. 14, 2024

(54) OCULAR DEVICE AND METHODS

(71) Applicant: Vinci Pharmaceuticals, Inc., Avon by the Sea, NJ (US)

(72) Inventors: Karl Csaky, Dallas, TX (US); Philip Gioia, Avon By The Sea, NJ (US)

(21) Appl. No.: 18/686,696

(22) PCT Filed: Aug. 26, 2022
(86) PCT No.: PCT/US2022/075538

§ 371 Date: Feb. 26, 2024

Related U.S. Application Data
(60) Provisional application No. 63/397,202, filed on Aug. 11, 2022.
(60) Provisional application No. 63/260,621, filed on Aug. 26, 2021.

(51) Int. Cl. A61F 2/14; A61L 27/54

(52) U.S. Cl. CPC A61F 2/148; A61L 27/54

(57) ABSTRACT

New multilayer ocular implant devices are provided. In certain aspects, the device is adapted for residence in the sub-Tenon's space of a patient's eye. In certain systems, separate layers of the implant device will contain distinct therapeutic agents, for example two or more different agents that can deliver desirable pharmacological effects, in certain preferred systems, the distinct therapeutic agents in separate device layers may provide a coordinated pharmacological effect, for example a first layer may contain a primary therapeutic agent and a second layer may contain a therapeutic agent that provides a supportive benefit to the primary therapeutic agent such as reducing side effects or enhancing patient uptake of the primary therapeutic agent or other delivery enhancement.

The present application claims priority from 1) U.S. provisional application No. 63/397,202 filed Aug. 11, 2022 and 2) U.S. provisional application No. 63/260,621 filed Aug. 26, 2021, which of which applications are incorporated herein by reference in its entirety.

### 1. FIELD

The present disclosure generally relates to local therapies for the eye and, more particularly, to ocular implant devices. In certain aspects, the implant device is adapted for residence into the sub-Tenon's space of a patient's eye.

### 2. BACKGROUND

In the treatment of many diseases and disorders of the eye, and especially in the case of degenerative or persistent conditions, implantable sustained-release delivery devices have been desired. See Gote et al, Journal of Pharmacology and Experimental Therapeutics, September 2019, 370 (3) 602-624.

Certain ocular implants and placements have been reported for dispensing a therapeutic drug to the eye. Particularly useful systems have been disclosed in U.S. Pat. No. 10,881,609.

It would be desirable to have new ocular implant devices and drug delivery systems.

### SUMMARY

New ocular implant devices are now provided that are useful for administration of therapeutic agents to a patient's eye.

In one aspect, the ocular implant devices include three or more layers, where each layer is distinct from an adjoining layer.

It has been found that such a multiple layer implant device can provide notable advantages, including enhanced delivery of a therapeutic agent. For instance, the multiple layers can enable desired delivery rates of the therapeutic agent to a patient.

In one preferred aspect, the ocular implant device includes three or more layers and one or more therapeutic agents are present in at least one layer of the device.

In certain preferred aspects, the ocular implant device includes three or more layers and one or more therapeutic agents are present in at least two or three layers of the device. The therapeutic agent(s) present in separate layers suitably may be the same or different.

In certain systems, separate layers of the implant device will contain distinct therapeutic agents, for example two or more different agents that can deliver desirable pharmacological effects. In certain preferred systems, the distinct therapeutic agents in separate device layers may provide a coordinated pharmacological effect, for example a first layer may contain a primary therapeutic agent and a second layer may contain a therapeutic agent that provides a supportive benefit to the primary therapeutic agent such as reducing side effects or enhancing patient uptake of the primary therapeutic agent or other delivery enhancement.

In particular preferred systems, at least one administered therapeutic agent may control or modulate lymphatic clearance or absorption of a distinct primary therapeutic being administered by the device. The primary therapeutic agent and the lymphatic adsorption modulator suitably may be contained in distinct layers of the implant device.

In additional particular preferred systems, at least one administered therapeutic agent may comprise a steroid or other agent (including a non-steroid agent) that controls fibrosis and is administered in conjunction with another distinct therapeutic agent. The anti-fibrotic agent and the additional distinct therapeutic agent suitably may be contained in distinct layers of the implant device.

In a preferred system, a multilayer ocular implant device is provided that comprises:
(a) a first layer comprising a polymer;
(b) a second layer 1) distinct from the first layer and 2) comprising one or more therapeutic agents, and
(c) a third layer. The implant device suitably may have one or more additional layers (four, five layers or more) that are distinct from at least one other layer.

In one system, a primary therapeutic agent may be loaded in an internal core layer that is encased within two or more distinct implant layers. A first layer abutting or adjoining a therapeutic agent core layer may be substantially impermeable to the therapeutic agent, providing directional delivery of the therapeutic agent such as to the opposing implant device side. A further layer abutting or adjoining a therapeutic agent core layer on an opposing therapeutic core layer suitably may comprise one or more materials that provide for extended and controllable release of the therapeutic agent from the core layer and through that second layer for administration to the patient. For instance, the further layer abutting a core therapeutic may comprise one or more polymers that may degrade over time or otherwise modulate release of a therapeutic agent. Such polymers may include any of a variety of biodegradable or bioerodable polymers e.g. poly(lactic-co-glycolic acid) (PLGA), poly(caprolactones) (PCL), poly(ethylene glycol) (PEG), polyethylene glycol diacrylate (PEGDA), poly(glycerol sebacate) (PGS) and/or poly (glycerol sebacate urethane) (PGSU).

In another multilayer implant device system, one or more therapeutic agents may be contained in two or more device layers. For instance, a core device layer may contain a primary therapeutic agent and a second layer adjoining the core layer may comprise a second, distinct therapeutic that may support the primary therapeutic of the core layer, e.g. to treat a possible side effect or provide desired bioavailability such as enhanced exposure of the primary therapeutic agent. Alternatively, an implant device core layer may comprise the second therapeutic and a more outer implant device layer may comprise the primary therapeutic being administered to the patient.

In preferred devices, suitably the implant device has a curved surface.

In preferred systems, the third layer comprises one or more rate-controlling agents and/or one or more therapeutic agents whereby the administration rate and duration of the one or more therapeutic agents can be extended for prolonged periods, including 1, 2 3, 4, 5, 6 or more days, or 1, 2 3, 4 or more weeks, or more extended periods, such as 1, 2, 3, 4, 5, 6, 7, 8, 9, 10, 12, 15, 18, 21, 24, 27, 30, 33 or 36 months or longer.

In certain preferred systems, the third layer is interposed between the first layer and second layer. In the preferred systems, the third layer is an outermost layer of the implant device.

The distinct layers of an implant device may have a variety of configurations. For instance, suitably, the cross-sectional thickness of the third layer is less than the cross-sectional thickness of the first layer and/or the cross-sectional thickness of the third layer is less than the cross-sectional thickness of the second layer.

Suitably, the third layer comprises a polymer such as a polyimide or other material, including one or more other materials that can inhibit transport of a therapeutic agent through the third layer.

In certain embodiments, the first layer does not contain a therapeutic agent, including where the first layer is an outermost layer of an implant device. Suitably, the second layer comprises a polymer distinct from the first layer polymer.

In certain preferred configurations, the first layer extends circumferentially beyond the second layer such that the surface of the circumferential extension of the first hardened layer is capable of making contact with the sclera of the eye. Preferably, at least one surface of the second layer is capable of making contact with the sclera of a subject's eye.

In certain preferred systems, an implant device may comprise a fourth layer distinct from each of the first, second or third layer.

In certain other preferred systems, an implant device comprises a fourth layer distinct from an adjacent layer.

In additional certain preferred systems, an implant device may comprise a fifth layer distinct from each of the first, second, third or fourth layer.

In additional preferred systems, the implant device comprises a fifth layer distinct from an adjacent layer.

In certain preferred aspects, an implant device will contain a single therapeutic agent. That therapeutic agent suitably may be present in a single layer of the multiple layer device. In certain aspects, each layer of a device may comprise a single type of polymer.

The multiple layer ocular implant device preferably comprises for administration to a subject one more non-steroidal anti-inflammatory drugs (NSAIDs), particularly one or more of bromfenac; diclofenac; indomethacin; nepafenac; metformin; flurbiprofen; suprofen; and/or ketorolac, or a pharmaceutically acceptable salt thereof.

In another aspect, the multiple layer ocular implant device preferably comprises N-acetylcysteine amide (NAC amide or NACA) for administration to a subject.

In another aspect, the multiple layer ocular implant device preferably comprises one or more prostaglandins such as latanoprost for administration to a subject.

The present ocular implant devices also may comprise one or more other therapeutic agents.

In one aspect, a layer (e.g. first layer) of the implant device will be considered distinct from another layer (e.g. second layer) where the two layers differ in composition and/or function. For instance, a first layer may contain one or more therapeutic agents and an adjoining layer that does not contain at least the same or any therapeutic agents will be a distinct layer as referred to herein. In such exemplary systems, the polymer matrix of those two layers may be the same or different.

Distinct layers also may contain one or more of the same polymers or other materials, but differ in at least one material such as at least one polymer. For example, an outer drug delivery-modulating layer may comprise an additional polymer that modulates release of a therapeutic agent from the implant device to the patient.

In other embodiment, two layers will be distinct where the two layers differ in function such as permeability of a therapeutic agent through the layer.

A single layer suitably also may not have a homogenous composition throughout the layer. For instance, a first portion of a layer's cross-sectional thickness may be relatively similar to an adjacent layer but a second portion of layer may have a more significant amount of a component such as polymer or therapeutic agent that is not present in the layer first portion.

Such a graded compositional profile across a layer cross-section can provide more precise device function such as extended drug delivery from the implant device.

Distinct device layers also may be fabricated in separate steps during production of an implant device. For instance, a device substrate may have a first layer composition applied such as by coating and in a separate and subsequent step a second layer composition may be applied such as by coating over the first layer or another other material that has been applied over the first layer. A single layer also may be produced in multiple steps, for example in multiple polymer deposition steps.

Thickness of each layer suitably may vary widely. Typical layer thickness may range from about 30 μm to 5 or 8 mm, more typically 30 μm to 0.5, 0.6, 0.7, 0.8, 0.9 or 1 or 2 mm or more. In certain aspects, a layer that comprises a therapeutic agent may have a comparatively greater thickness than an adjoining layer that does not contain a therapeutic agent. In certain aspects, the one or more layers of an implant device are each about 2 mm or less thick, such as 1.9, 1.8, 1.7, 1.6, 1.5, 1.4, 1.3, 1.2, 1.1 1, 0.9. 0.8. 0.7, 0.6, 0.5, 0.4, 0.3 or 0.2 mm or less thick. For layer(s) that comprise therapeutic agent, the layer thickness may be dictated at least in part by the therapeutic agent's solubility and the amount of therapeutic agent needed to achieve targeted release duration.

As discussed, the present implant devices preferably have a curved surface. In preferred configurations, the implant device is circular or oval shape. In additional preferred configurations, an implant device has a configuration that enhances anchoring upon implanting to a patient. For instance, the implant device may comprise a feature that permits attachment of a suture to the device.

In one aspect, preferably at least one layer of an implant device will comprise a biodegradable or bioerodable material, such as a polymer material that degrades over time in vivo. In certain preferably multiple or all layers of an implant device may comprise one or more biodegradable or bioerodable materials.

In one embodiment, an outer layer (i.e. an implant device layer that has an extended cross-sectional surface exposed or otherwise in direct contact with patient tissue) will comprise a biodegradable or bioerodable material.

Exemplary biodegradable or bioerodable polymers that may be incorporated into an implant device layer comprise for example poly(lactic-co-glycolide), polylactic-polyglycolic acid polymers (PLGA), hydroxypropyl methyl cellulose, hydroxyl methyl cellulose, polyglycolide-polyvinyl alcohol, croscarmellose sodium, hydroxypropylcellulose, carboxymethylcellulose (e.g. sodium carboxymethylcellulose), polyglycolic acid-polyvinyl alcohol copolymers (PGA/PVA), hydroxypropylmethylcellulose (HPMC), poly(glycerol sebacate) (PGS), poly (glycerol sebacate urethane) (PGSU) and/or polycaprolactonepolyethylene glycol polymer materials.

In one aspect, preferably at least one layer of an implant device comprises a polylactic-polyglycolic acid (PLGA) material.

In one aspect, preferably at least one layer of an implant device comprises a polylactic-polyglycolic acid (PLGA) material and that device layer comprises one or more therapeutic agents.

In one aspect, preferably at least one layer of an implant device comprises a poly(glycerol sebacate) (PGS) material.

In one aspect, preferably at least one layer of an implant device comprise a poly(glycerol sebacate) (PGS) material and that device layer comprises one or more therapeutic agents.

In one aspect, preferably at least one layer of an implant device comprises a poly(glycerol sebacate urethane) (PGSU) material.

In one aspect, preferably at least one layer of an implant device comprises a poly(glycerol sebacate urethane) (PGSU) material and that device layer comprises one or more therapeutic agents.

In the present implant devices, suitably at least one layer is at least substantially impermeable to diffusion of therapeutic agent. Such a layer can direct diffusion of a therapeutic agent to the patient. For instance, the at least one impermeable layer may comprise one or more polymers of polyvinyl acetate, cross-linked polyvinyl alcohol, cross-linked polyvinyl butyrate, ethylene ethylacrylate co-polymer, polyethyl hexylacrylate, polyvinyl chloride, polyvinyl acetals, ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumarate copolymer, silicone rubbers, medical grade polydimethylsiloxanes, ethylene-propylene rubber, silicone-carbonate copolymers, vinylidene chloride-vinyl chloride copolymer, vinyl chloride-acrylonitrile copolymer or vinylidene chloride-acrylonitride copolymer.

Suitably, at least one implant device layer may comprise one or more of polyvinyl acetate, cross-linked polyvinyl alcohol, cross-linked polyvinyl butyrate, ethylene ethylacrylate co-polymer, polyethyl hexylacrylate, polyvinyl chloride, polyvinyl acetals, plasticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumarate copolymer, thermoplastic polyurethane (TPU), silicone rubbers, medical grade polydimethylsiloxanes, ethylene-propylene rubber, silicone-carbonate copolymers, vinylidene chloride-vinyl chloride copolymer, vinyl chloride-acrylonitrile copolymer or vinylidene chloride-acrylonitride copolymer.

A preferred material for an outer implant device layer is a polyethylene including LDPE (low density polyethylene).

Preferred materials for an implant device layer that comprises one or more therapeutic agents include vinyl acetate and ethylene-vinyl acetate (EVA). It also can be preferred that a bioerodable device layer (e.g. an implant layer that comprises poly(glycerol sebacate urethane) (PGSU) comprises one or more therapeutic agents.

In one aspect, at least one implant device layer comprises a permeability enhancer that increases ocular permeability of the therapeutic agent into the eye.

In one aspect, an implant device layer may comprise a therapeutic agent admixed with a controlled release composition. Suitable controlled release compositions may comprise one or more polymers, such as one or more biodegradable polymers, where the one or more biodegradable polymers may degrade over extended time in a patient eye to thereby administer the one or more therapeutic agents to the patient. Exemplary biodegradable polymers that may be incorporated into an implant device layer comprise for example poly(lactic-co-glycolide), polylactic-polyglycolic acid block copolymers (PLGA), hydroxypropyl methyl cellulose, hydroxyl methyl cellulose, polyglycolide-polyvinyl alcohol, croscarmellose sodium, hydroxypropylcellulose, sodium carboxymethylcellulose, polyglycolic acid-polyvinyl alcohol block copolymers (PGA/PVA), hydroxypropylmethylcellulose (HPMC), poly(glycerol sebacate) (PGS), poly (glycerol sebacate urethane) (PGSU) and/or polycaprolactonepolyethylene glycol block copolymers.

A bioerodable layer also may be configured to provide a desired release rate by selection of cross-linking densities of a polymer matrix of the layer, choice of layer materials and layer thickness, among others. A desired degradation rate of a layer intended to erode over a particular duration can readily be identified empirically by testing of implant device layers with varying materials, cross-linking density and layer thickness, among other characteristics.

In further aspect, methods are provided for delivery of a therapeutic agent to a patient's eye that comprise: (a) providing an implant device as disclosed herein wherein the device comprises one or more therapeutic agents; and (b) inserting the device into a patient's eye.

Preferably, the implant device is placed into the sub-Tenon's space and in contact with the sclera of the eye.

The patient may be treated for a variety of disorders and diseases including macular degeneration and in particular age-related macular degeneration (AMD).

In certain aspects, the patient may be treated for diabetic macular edema (DME). In one embodiment, a patient may be identified as suffering from or susceptible to diabetic macular edema which may include accumulation of excess fluid in the extracellular space within the retina in the macular area, and the identified patient treated with an implant as disclosed herein.

In certain aspects, the patient may be treated for cystoid macular edema or CME. In one embodiment, a patient may be identified as suffering from or susceptible to cystoid macular edema, and the identified patient treated with an implant as disclosed herein.

Treatment kits also are provided and may comprise (a) an implant device as disclosed herein and (b) instructions for use of the implant device to treat an eye disorder.

A treatment kit suitably may include other components.

Further provided are methods for evaluating the effectiveness of the implant device as described herein for treatment or prevention of an ocular disease or disorder such as macular degeneration in a human, the method comprising: a) placing the implant device as described herein into the sub-Tenon's space of the eye of the human; and b) examining the eye of the human using a suitable analysis technique, thereby evaluating the effectiveness of the implant device against an evaluated ocular disease or disorder.

Any of a variety of analysis or interrogation procedures may be utilized, for example 2 color (blue, red) microperimetry, low luminance visual acuity, multi-focal electroretinography, dynamic perimetry, color vision assessment, photo-stress testing and static perimetry, contrast sensitivities, qCST, BCVA, qVA, OCT/FAF/IR/OCT-A/Flavo-protein detection/color fundus photos and others.

Other aspects of the invention are disclosed infra.

### BRIEF DESCRIPTION OF THE DRAWING

FIG. 1 depicts schematically a preferred multilayer implant device.

FIG. 2 depicts schematically a further preferred multiplayer implant device.

FIG. 3 is a photograph of an implant of Example 2 which follows.

### DETAILED DESCRIPTION

As discussed, in various aspects, a composite ocular implant device is provided comprising a therapeutic agent for treatment or prevention of a disorder of the eye. In preferred systems, implant device provides sustained release of the therapeutic agent during the treatment or prevention of the disorder of the eye. This device configuration is particularly well-suited for placement in the sub-Tenon's space (also known as the bulbar sheath) but is not limited thereto and could be installed on or in other eye regions where convenient and useful.

The present implant devices can be used to treat a number of eye diseases and indications including, for example, age-related macular degeneration, glaucoma, diabetic retinopathy, uveitis, retinopathy of prematurity in newborns, choroidal melanoma, chorodial metastasis, and retinal capillary hemangioma.

### Multiple-Layer Ocular Implant Device

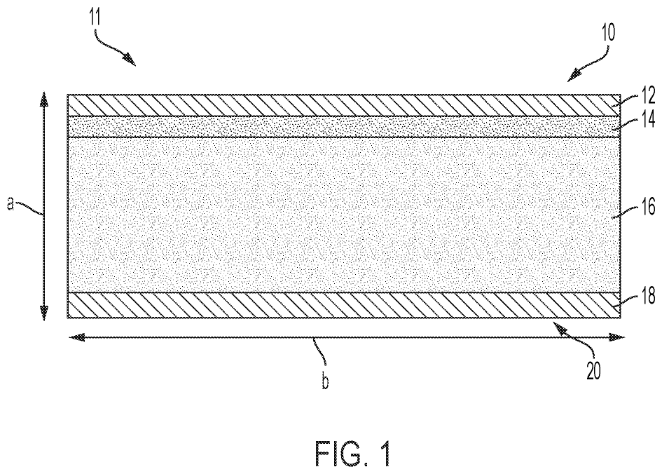

Referring now to the drawings, FIG. 1 shows schematically a preferred ocular implant device 10 which includes multiple layers 12, 14, 16 and 18. The implant device also suitably may contain additional layers.

Suitably, one or more layers comprises one or more therapeutic agents such as a nonsteroidal anti-inflammatory drugs (NSAIDs), or other agent including those therapeutic agents disclosed below.

In certain systems, an outmost layer such as layer 12 or 18 depicted in FIG. 1 may function as a drug release rate limiting membrane. For example, the layer may be a polymer matrix (including a cross-linked matrix) that can modulate flow of one or more therapeutic agents through the layer and out of the device to scleral surface 11.

In one configuration, layer 14 may comprise one or more therapeutic agents. The layer suitably comprises one or more polymers in which the therapeutic agent(s) are admixed. In certain aspects, it may be preferred that an implant device layer contains a single polymer rather than a blend of two or more distinct polymers.

In one configuration, layer 12 may function as a drug diffusion barrier layer. For example, the layer may comprise a polymer or polymer matrix that substantially impedes passage of the therapeutic agent(s) present in layer 14 through the layer 16.

In one configuration, layer 18 may be a further polymer layer. In one aspect, layer 18 may contain one or more therapeutic agents which may be the same or distinct from the one or more therapeutic agents present in layer 14. As discussed above, in certain preferred aspects one or more therapeutic agents of layer 18 will be distinct from therapeutic agents present in layer 14. Layer 18 therapeutic agents suitably may be administered to Tenon's/conjunctiva surface 20.

In other aspects, layer 18 may not contain a therapeutic agent.

In other preferred systems, an encased or inner layer such as layer 14 and/or 16 depicted in FIG. 1 may comprise one or more therapeutic agents. In such configurations, the outer layers 12 and/18 depicted in FIG. 1 can function to control drug administration such as by modulating release of a therapeutic agent from layer 14 or 16 to a subject. For instance, layer 12 and/or 18 may comprise one or more bioerodable or biodegradable materials that will release therapeutic agent(s) from the implant device over extended time such as 1, 2, 3, 4, 5, 6, or 7 days or more, or periods such as 2, 3, 4, 5, 6, 7, or 8 weeks or more, or more extended periods as discussed above, such as 0.5 year, 1 year, 1.5 years, 2 years, 2.5 years, 3 years or more.

The various layers 12, 14, 16, 18 may comprise one or more polymers such as polyvinyl acetate, cross-linked polyvinyl alcohol, cross-linked polyvinyl butyrate, ethylene ethylacrylate co-polymer, polyethyl hexylacrylate, polyvinyl chloride, polyvinyl acetals, plasticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumarate copolymer, silicone rubbers, medical grade polydimethylsiloxanes, ethylene-propylene rubber, silicone-carbonate copolymers, vinylidene chloride-vinyl chloride copolymer, vinyl chloride-acrylonitrile copolymer or vinylidene chloride-acrylonitride copolymer or any suitable equivalent of these polymers or combinations thereof.

In certain systems, a vinyl acetate or ethylene-vinyl acetate (EVA) material is a preferred material for one or more implant device layers.

Dimensions of an implant device may vary. However, in this particular embodiment, the implant device 10 has a diameter (shown as dimension b in FIG. 1) of 7 mm and a thickness (shown as dimension a in FIG. 1) of 2 mm. In this particular embodiment, each of the two layers 16 and 18 is 1 mm thick. As discussed, layers 16 and 18 suitably may be a wide ranges of thicknesses, including for instance 0.5 mm to 25 mm. In this particular embodiment, the outer surface 18′ of layer 18 has a radius of curvature of 1 cm or less for generally conforming to the radius of curvature of the surface of Tenon's capsule of an average human eye. Likewise, layer 12 is also curved with a similar radius of curvature configured to generally conform to the radius of curvature of the sclera of an average human eye. These dimensions provide the implant device 10 with characteristics appropriate for implantation with scleral contact in the sub-Tenon's space of a human. It will be understood by the skilled person that these dimensions should be modified appropriately for an implant device designed for use in an experimental animal such as a rat, mouse or rabbit for example. Armed with the knowledge of average dimensions of the eye and radii of curvature of Tenon's capsule and sclera of the chosen experimental animal, the dimensions of an ocular implant device according to may be selected by the skilled person and appropriate molding tools may be constructed without undue experimentation.

In certain systems, it can be advantageous to provide the ocular implant device with an upper layer 12 and/or 14 as shown in FIG. 1 which is generally resistant to diffusion of the therapeutic agent which is dispersed in the layer 16. In certain embodiments, the layer 12 is impermeable to the therapeutic agent. In other embodiments, the therapeutic agent has a rate of diffusion within layer 12 which is greater than the rate of diffusion of the therapeutic agent through layer 16. The differential diffusion characteristics of the therapeutic agent through specific implant device layer provide the advantage of preventing loss of the therapeutic agent to tissues where it is not needed. For instance, the reduced rate of diffusion of the therapeutic agent through layer 16 encourages unidirectional diffusion of the therapeutic agent into the sclera and choroid for transfer to the macula. A further advantage provided by the reduced diffusion characteristics of the therapeutic agent in the layers 12 and/or 14 relative to the layer 16 or 18 (18 if present) is gained in preventing the therapeutic agent from entering the lymphatic system via Tenon's capsule and the conjunctiva for transfer to other tissues where it may cause undesirable side-effects. Thus, in certain embodiments of the present disclosure, as discussed above, one or more layers of the implant device further includes an agent that blocks lymphatic absorption.

As further discussed above, distinct implant device layers may comprise other coordinated multiple drug therapies. For instance, as discussed, one layer may comprise an antifibrotic such as a steroid or 5-fluoruracil or mitomycin C and suitably a separate layer will contain one or more additional distinct therapeutic agents.

In a particular embodiment, the administered therapeutic agent in layer 14 is a non-steroidal anti-inflammatory drug (NSAID) such as nepafenac. The drug is released through diffusion through layer 12 depicted in FIG. 1.

In a particular preferred embodiment, implant device 10 is located in the sub-Tenon's space of a patient's eye with an implant device surface resting upon the surface of the sclera. Preferably, an opposed upper implant device layer has a curvature which generally conforms to the curvature of the surface of Tenon's capsule. Such a configuration can minimize discomfort to the eye as a result of contact of Tenon's capsule with upper edges of the implant device 10. In certain preferred configurations, a curved upper surface of the implant device is smooth and does not have sharp edges which would otherwise cause irritations and/or damage to the tissues of Tenon's capsule and possibly also the conjunctiva in the event that a sharp edge of an alternative implant were to completely puncture Tenon's capsule and penetrate the conjunctiva.

In certain preferred systems, one or more therapeutic agents contained in the implant device will be released to the sclera because the therapeutic agent(s) are concentrated in the implant device layer 14 and because the upper layer 12 allows diffusion of the therapeutic agent. In certain systems, therapeutic agent(s) administered by an implant device may be transferred by either diffusion or an active physiological mechanism, or a combination thereof, to the macula where the desired pharmaceutical effect will be obtained.

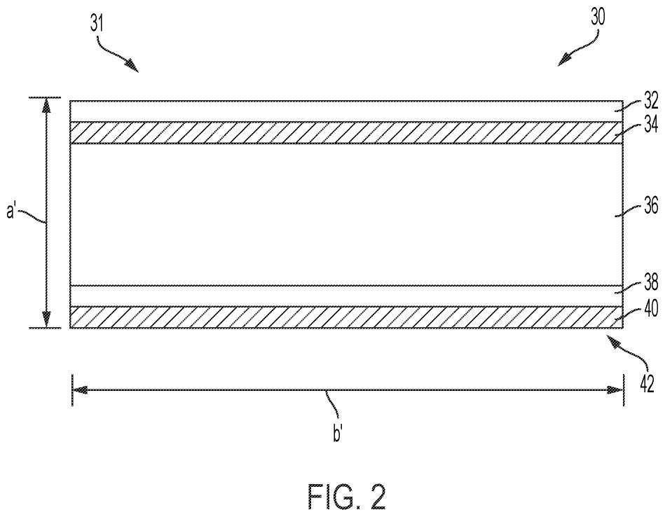

A further preferred implant device 30 is show in FIG. 2 which contains at least five distinct implant device layers. Thus, implant device 30 in FIG. 2 includes layers 32, 34, 36, 38 and 40. Any of the layers may include one or more therapeutic agents. One of more layers can function to control drug administration such as by modulating release of a therapeutic agent as discussed above, through one or more bioerodable or biodegradable materials that will release therapeutic agent(s).

In one preferred system, device 30 includes drug release rate limiting membrane 32 that contacts scleral surface 31. For example, layer 32 may be a polymer matrix (including a cross-linked matrix) that can modulate flow of one or more therapeutic agents through the layer and out of the device to scleral surface 31.

In one configuration, layer 36 may comprise one or more therapeutic agents. The layer suitably comprises one or more polymers in which the therapeutic agent(s) are admixed. In one configuration, layer 32 or 34 may function as a drug diffusion barrier layer. For example, the layer may comprise a polymer or polymer matrix that substantially impedes passage of the therapeutic agent(s) present in layer 36 through the layer 32.

In one configuration, layer 38 may be a further polymer layer. In one aspect, layer 38 may contain one or more therapeutic agents which may be the same or distinct from the one or more therapeutic agents present in layer 36. As discussed above, in certain preferred aspects one or more therapeutic agents of layer 38 will be distinct from therapeutic agents present in layer 36. Layer 38 therapeutic agents suitably may be administered to Tenon's/conjunctiva surface 42.

In one configuration, layer 40 is a drug release rate limiting membrane that contacts Tenon's/conjunctiva surface 42. Thus, for example, layer 40 may be a polymer matrix (including a cross-linked matrix) that can modulate flow of one or more therapeutic agents through the layer and out of the device to scleral surface.

### Therapeutic Agents

As discussed, an implant device may comprise and administer a variety of therapeutic agents to a subject.

In one aspect, one or more therapeutic agents that may be administered to a patient with a present implant device include, but are not limited to: antibiotic agents such as fumagillin analogs, minocycline, fluoroquinolone, cephalosporin antibiotics, herbimycon A, tetracycline, chlortetracycline, bacitracin, neomycin, polymyxin, gramicidin, oxytetracycline, chloramphenicol, gentamicin and erythromycin; antibacterial agents such as sulfonamides, sulfacetamide, sulfamethizole, sulfoxazole, nitrofirazone, and/or sodium propionate.

In another aspect, one or more therapeutic agents that may be administered to a patient with a present implant device include N-acetyl cysteine (NAC) alkyl-ester analogue compounds, including compounds disclosed in WO2021/092470.

In another aspect, one or more therapeutic agents that may be administered to a patient with a present implant device include N-acetylcysteine amide (NAC amide or NACA).

In another aspect, one or more therapeutic agents that may be administered to a patient with a present implant device include one or more prostaglandins such as latanoprost.

The present ocular implant devices also may comprise one or more

In another aspect, one or more therapeutic agents that may be administered to a patient with a present implant device include antiviral agents such as idoxuridine, famvir, trisodium phosphonoformate, trifluorothymidine, acyclovir, ganciclovir, DDI and AZT, protease and/or integrase inhibitors.

In another aspect, one or more therapeutic agents that may be administered to a patient with a present implant device include anti-glaucoma agents such as beta blockers (timolol, betaxolol, atenolol), prostaglandin analogues, hypotensive lipids, and/or carbonic anhydrase inhibitors.

In another aspect, one or more therapeutic agents that may be administered to a patient with a present implant device include antiallergenic agents such as antazoline, methapyriline, chlorpheniramine, pyrilamine and/or prophenpyridamine.

In another aspect, one or more therapeutic agents that may be administered to a patient with a present implant device include anti-inflammatory agents such as hydrocortisone, leflunomide, dexamethasone phosphate, fluocinolone acetonide, medrysone, methylprednisolone, prednisolone phosphate, prednisolone acetate, fluoromethalone, betamethasone, triamcinolone acetonide, adrenalcortical steroids and their synthetic analogues, and/or 6-mannose phosphate.

In another aspect, one or more therapeutic agents that may be administered to a patient with a present implant device include antifungal agents such as fluconazole, amphotericin B, liposomal amphotericin B, voriconazole, imidazole-based antifungals, tiazole antifungals, echinocandin-like lipopeptide antibiotics, lipid formulations of antifungals; polycations and polyanions such as suramine and/or protamine.

In another aspect, one or more therapeutic agents that may be administered to a patient with a present implant device include decongestants such as phenylephrine, naphazoline, and/or tetrahydrazoline.

In another aspect, one or more therapeutic agents that may be administered to a patient with a present implant device include anti-angiogenesis compounds including those that can be potential anti-choroidal neovascularization agents such as 2-methoxyestradiol and its analogues (e.g., 2-propynl-estradiol, 2-propenyl-estradiol, 2-ethoxy-6-oxime-estradiol, 2-hydroxyestrone, 4-methoxyestradiol), VEGF antagonists such as VEGF antibodies and VEGF antisense, angiostatic steroids (e.g., anecortave acetate and its analogues, 17-ethynylestradiol, norethynodrel, medroxyprogesterone, mestranol, androgens with angiostatic activity such as ethisterone), thymidine kinase inhibitors.

In another aspect, one or more therapeutic agents that may be administered to a patient with a present implant device include adrenocortical steroids and their synthetic analogues including fluocinolone acetonide and triamcinolone acetonide and all angiostatic steroids.

In another aspect, one or more therapeutic agents that may be administered to a patient with a present implant device include immunological response modifying agents such as cyclosporineA, Prograf (tacrolimus), macrolide immunosuppressants, mycophenolate mofetil, rapamycin, and muramyl dipeptide.

In another aspect, one or more therapeutic agents that may be administered to a patient with a present implant device include one or more anti-cancer agents such as 5-fluorouracil, platinum coordination complexes such as cisplatin and carboplatin, adriamycin, antimetabolites such as methotrexate, anthracycline antibiotics, antimitotic drugs such as paclitaxel and docetaxel, epipdophylltoxins such as etoposide, nitrosoureas including carmustine, alkylating agents including cyclophosphamide; arsenic trioxide; anastrozole; tamoxifen citrate; triptorelin pamoate; gemtuzumab ozogamicin; irinotecan hydrochloride; leuprolide acetate; bexarotene; exemestrane; epirubicin hydrochloride; ondansetron; temozolomide; topoteanhydrochloride; tamoxifen citrate; irinotecan hydrochloride; trastuzumab; valrubicin; gemcitabine; goserelin acetate; capecitabine; aldesleukin; rituximab; oprelvekin; interferon alfa-2a; letrozole; toremifene citrate; mitoxantrone hydrochloride; irinotecan; topotecan; etoposide phosphate; gemcitabine; and amifostine; antisense agents; antimycotic agents; miotic and anticholinesterase agents such as pilocarpine, eserine salicylate, carbachol, diisopropyl fluorophosphate, phospholine iodine, and demecarium bromide; mydriatic agents such as atropine sulfate, cyclopentane, homatropine, scopolamine, tropicamide, eucatropine, and hydroxyamphetamine; differentiation modulator agents; sympathomimetic agents such as epinephrine; anesthetic agents such as lidocaine and benzodiazepam; vasoconstrictive agents; vasodilatory agents; polypeptides and protein agents such as angiostatin, endostatin, matrix metalloproteinase inhibitors, platelet factor 4, interferon-gamma, insulin, growth hormones, insulin related growth factor, heat shock proteins, humanized antilL2 receptor mAb (Daclizumab), etanercept, mono and polyclonal antibodies, cytokines, antibody to cytokines.

In another aspect, one or more therapeutic agents that may be administered to a patient with a present implant device include neuroprotective agents such as calcium channel antagonists including nimodipine and diltiazem, neuroimmunophilin ligands, neurotropins, memantine and other NMDA antagonists.

In another aspect, one or more therapeutic agents that may be administered to a patient with a present implant device include acetylcholinesterase inhibitors, estradiol and analogues, vitamin B12 analogues, alpha-tocopherol, NOS inhibitors, antioxidants (e.g. glutathione, superoxide dismutase), metals like cobalt and copper, neurotrophic receptors (Akt kinase), growth factors, nicotinamide (vitamin B3), alpha-tocopherol (vitamin E), succinic acid, dihydroxylipoic acid, fusidic acid; cell transport/mobility impending agents such as colchicine, vincristine, cytochalasin B; carbonic anhydrase inhibitor agents; integrin antagonists; lipophilic agents such as Idebenone, rapamycin, 2-cyano-3, 12 dioxooleana-1,9 dien-28-imidazolide (CDDO-Im), 2-cyano-3,12-dioxooleana-1,9(11)-dien-28-oic acid-ethyl amide (CDDO-ethyl amide), and 2-cyano-3,12-dioxooleana-1,9(11)-dien-28-oic acid trifluoroethyl amide (CDDO-TFEA); and lubricating agents.

In particular aspects, preferred therapeutic agents to be administered to a patient's eye with a multiplayer implant device as disclosed include one more non-steroidal anti-inflammatory drugs (NSAIDs), for example one or more of bromfenac; diclofenac; indomethacin; nepafenac; metformin; N-acetylcysteine amide (NAC amide); flurbiprofen; suprofen; and/or ketorolac, or a pharmaceutically acceptable salt thereof.

Any pharmaceutically acceptable form of the agents can be used, such as the free base form or a pharmaceutically acceptable salt or ester thereof. In this particular embodiment, the dosage of the therapeutic agent provided by the present implant device is in the range of 1-100 mg.

### Administration of or Using the Multiple-Layer Ocular Implant Device

In one preferred aspect to administer the implant device, the subconjunctival matrix implant device preferably is placed behind the surface epithelium within the sub-Tenon's space. This is done by a surgical procedure that can be performed in an out-patient setting. A lid speculum is placed and a conjunctival radial incision is made through the conjunctiva over the area where the implant device is to be placed. Wescott scissors are used to dissect posterior to Tenon's fascia and the implant device is inserted. The conjunctiva is reapproximated using a running 10-0 vicryl suture. The eye has many barriers that do not permit easy penetration of drugs. These include the surface epithelium on the front (cornea) of the eye and the blood/retinal barrier either within the retinal blood vessels or between the retinal pigment epithelium that both have tight junctions. These implant devices are generally about 1-2 mm in diameter for small rodent (i.e., mouse and rat) eyes, 3-4 mm in diameter for rabbit and human eyes and 6-8 mm in diameter for equine eyes.

In certain embodiments, an applicator device is used to inject the implant device into the sub-Tenon's space. Such devices are known in the art and have been used for intraocular injections into the vitreous humor of the eye, particularly in intraocular lens implantation after cataract surgery. In certain embodiments, the device is provided with a retractor that engages the conjunctiva and the surface of Tenon's capsule to produce an opening into the sub-Tenon's space. The device is also provided with a means for pushing the implant device into the sub-Tenon's space such that withdrawal of the device allows the surrounding tissues to collapse back into place while holding the implant device at the desired location.

Additionally, when the implant device is placed near the limbus (i.e., the area where the conjunctiva attaches anteriorly on the eye) to encourage the drug diffusion to enter the cornea, it may be preferable to fixate the matrix implant device with one or two absorbable sutures (e.g., 10-0 absorbable vicryl sutures). This may be done by making holes with a 30 gauge needle in the peripheral portion of the implant, approximately 250-500 μm away from the peripheral edge of the implant.

The holes are made 180 degrees from each other. This is done because certain subconjunctival matrix implant devices of this disclosure, when placed near the cornea, are at higher risk to extrude because of the action of the upper eye lid when blinking. When subconjunctival matrix implant devices as disclosed herein are placed about 4 mm or more away from the limbus, the sutures are optional.

In addition to sutures, other attachment means can be employed including for example a biocompatible adhesive to adhere the implant device to a target site.

This matrix implant device can deliver therapeutic levels of different pharmaceuticals agents to the eye to treat a variety of diseases. Using a rabbit model, drug released from the implant placed in the eye produces negligible levels of the drug in the blood. This significantly reduces the chances of systemic drug side-effects. This implant device design of this disclosure is prepared by unique methodologies and selections of materials leading to and imparting the unique pharmacological performance properties present in the finished devices.

### Lipophilic Agents

In accordance with the present disclosure, the therapeutic agent or component of the implant device may comprise, consists essentially of, or consists of, a lipophilic agent. Such lipophilic agents may be small molecules. Lipophilic agents may be released from the implant device by diffusion, erosion, dissolution or osmosis. The drug release sustaining component may comprise one or more biodegradable polymers or one or more non-biodegradable polymers.

In one embodiment, the intraocular implant devices comprise a lipophilic agent. Lipophilic agents or other agent which may be employed in the implant devices include those disclosed in US Patent Publication, US20140031408, the contents of which are incorporated herein by reference in its entirety.

In another embodiment, intraocular implant devices comprise a therapeutic agent or component that comprises a lipophilic agent.

In preferred systems, the present implant devices provide a sustained or controlled delivery of therapeutic agents at a maintained level despite the rapid elimination of the lipophilic agents from the eye. The controlled delivery of lipophilic agents from the present implant devices permits the lipophilic agents to be administered into an eye with reduced toxicity or deterioration of the blood-aqueous and blood-retinal barriers, which may be associated with intraocular injection of liquid formulations containing lipophilic agents.

The present implant devices may be placed in an ocular region to treat a variety of ocular conditions, such as treating, preventing, or reducing at least one symptom associated with non-exudative age related macular degeneration, exudative age related macular degeneration, choroidal neovascularization, acute macular neuroretinopathy, cystoid macular edema, diabetic macular edema, Behcet's disease, diabetic retinopathy, retinal arterial occlusive disease, central retinal vein occlusion, uveitic retinal disease, retinal detachment, trauma, conditions caused by laser treatment, conditions caused by photodynamic therapy, photocoagulation, radiation retinopathy, epiretinal membranes, proliferative diabetic retinopathy, branch retinal vein occlusion, anterior ischemic optic neuropathy, non-retinopathy diabetic retinal dysfunction, retinitis pigmentosa, ocular tumors, ocular neoplasms, and the like.

Kits in accordance with the present disclosure may comprise one or more of the present implant devices, and instructions for using the implant devices. For example, the instructions may explain how to administer the implant devices to a patient, and types of conditions that may be treated with the implant devices. The kits also may further comprise for example an injector device or other applicator tools.

The present implant devices can be readily fabricated. Exemplary preferred fabrication protocols are set forth in the examples which follow. Injection molding and compression molding are preferred fabrication methods.

In general, a desired implant device layer may be prepared, for example a polymer matrix having a desired distribution of one or more therapeutic agents. That admixture may be applied such as by coating onto a substrate which may be a removable substrate if the admixture is a first layer being deposited, or the admixture may be applied over one or more previously applied layers. A mold may be utilized in forming the implant device. The device composite can be cured under heat and/or pressure in one or more steps such as in an injection molding or compression molding process.

### General Definitions

The following general definitions are supplied in order to facilitate the understanding of the present disclosure.

As used herein the term “radius of curvature” refers to the radius of a circle that best fits the curved surface at a given point.

As used herein, the term “permeation agent” refers to a molecule that increases the permeability of a therapeutic agent. An ophthalmic permeation agent increases the permeability of a therapeutic agent with respect to tissues of the eye.

As used herein the term “ophthalmic permeation agent” (also known as “transport facilitator”) refers to a compound that increases the permeability of a therapeutic agent into the tissues of the eye. Methylsulfonylmethane is a non-limiting example of an ophthalmic permeation agent.

As used herein, the term “microperimetry” refers to a technique which is used to assess the visual function of a specific area of the retina and fovea. It provides a quantifiable way to measure the regression or progression of retinal visual function in the examined eye. A dot of light is projected onto the retina at a specific intensity and the patient is asked to confirm reception of the light. Changes in the stimulus intensity followed by the patient response, provides the means to assess the retinal visual function. Variations in microperimetry include dynamic perimetry, two color (red and blue) perimetry and static perimetry and are known to those skilled in the art.

As used herein, the term “multi-focal electroretinography” refers to a technique for determining the activity of retinal cells. When bioelectrical changes occur within the retina, the change is propagated to the surface of the cornea. These small (and often very fast) signals can be captured by an electrode placed on the surface of the cornea. The subject fixates on the center of a display containing an array of hexagons that increase in size from the center outward.

Because the number of cone photoreceptors per area varies for different parts of the retina, the size of the hexagons is adjusted, so about the same number of cones will be stimulated by each hexagon. While the subject views the display, a single continuous electroretinogram recording is obtained.

As used herein, an “intraocular implant” refers to a device or element that is structured, sized, or otherwise configured to be placed in an eye. Intraocular implants are generally biocompatible with physiological conditions of an eye and do not cause adverse side effects. Intraocular implant devices may be placed in an eye without disrupting vision of the eye.

As used herein, an “ocular region” or “ocular site” refers generally to any area of the eyeball, including the anterior and posterior segment of the eye, and which generally includes, but is not limited to, any functional (e.g., for vision) or structural tissues found in the eyeball, or tissues or cellular layers that partly or completely line the interior or exterior of the eyeball. Specific examples of areas of the eyeball in an ocular region include the anterior chamber, the posterior chamber, the vitreous cavity, the choroid, the suprachoroidal space, the conjunctiva, the subconjunctival space, the episcleral space, the intracorneal space, the epicorneal space, the sclera, the pars plana, surgically-induced avascular regions, the macula, and the retina.

As used herein, an “ocular condition” is a disease, ailment or condition which affects or involves the eye or one of the parts or regions of the eye. Broadly speaking the eye includes the eyeball and the tissues and fluids which constitute the eyeball, the periocular muscles (such as the oblique and rectus muscles) and the portion of the optic nerve which is within or adjacent to the eyeball.

An “anterior ocular condition” is a disease, ailment or condition which affects or which involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves the conjunctiva, the cornea, the anterior chamber, the iris, the posterior chamber (behind the retina but in front of the posterior wall of the lens capsule), the lens or the lens capsule and blood vessels and nerve which vascularize or innervate an anterior ocular region or site. Thus, an anterior ocular condition can include a disease, ailment or condition, such as for example, aphakia; pseudophakia; astigmatism; blepharospasm; cataract; conjunctival diseases; conjunctivitis; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

A “posterior ocular condition” is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular region or site. Thus, a posterior ocular condition can include a disease, ailment or condition, such as for example, acute macular neuroretinopathy; Behcet's disease; choroidal neovascularization; diabetic uveitis; histoplasmosis; infections, such as fungal or viral-caused infections; macular degeneration, such as acute macular degeneration, non-exudative age related macular degeneration and exudative age related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, and glaucoma. Glaucoma can be considered a posterior ocular condition because the therapeutic goal is to prevent the loss of or reduce the occurrence of loss of vision due to damage to or loss of retinal cells or optic nerve cells (i.e. neuroprotection).

The term “biodegradable polymer” refers to a polymer or polymers which degrade in vivo, and wherein degradation of the polymer or polymers over time occurs concurrent with or subsequent to release of the therapeutic agent. Specifically, hydrogels such as methylcellulose which act to release drug through polymer swelling are specifically excluded from the term “biodegradable polymer”. The terms “biodegradable” and “bioerodible” are equivalent and are used interchangeably herein. A biodegradable polymer may be a homopolymer, a copolymer, or a polymer comprising more than two different polymeric units.

The term “treat”, “treating”, or “treatment” as used herein, refers to reduction or resolution or prevention of an ocular condition, ocular injury or damage, or to promote healing of injured or damaged ocular tissue.

The term “therapeutically effective amount” as used herein, refers to the level or amount of agent needed to treat an ocular condition, or reduce or prevent ocular injury or damage without causing significant negative or adverse side effects to the eye or a region of the eye.

The following non-limiting examples are illustrative.

### Example 1

An implant device as generally depicted in FIG. 1 is prepared as follows.

A drug layer composition is prepared by admixing a drug compound (nepafenac or other therapeutic agent) with ethylene-vinyl acetate (EVA). The admixture is heated at 70° C. for approximately 30 minutes.

The EVA-therapeutic agent composition then is placed in a mold that can apply heat and pressure and establish the device shape. The EVA-therapeutic agent composition layer in the configuration of the implant device is then removed from the mold and cooled. A vinyl acetate film is placed on opposing sides of the EVA-therapeutic agent layer. An interposing adhesive material may be used to secure the layers. A silicone adhesive (e.g. BIO-PSA 7-4302, DOW CORNING) is one suitable material.

The resulting multi-layer implant device suitably is 8 to 10 mm in diameter (dimension b in FIG. 1) and has an overall thickness of 1.8 to 2.0 mm (Dimension a in FIG. 1). The implant device suitably has an outer layer that is a 0.065 mm EVA film which is bonded to a LDPE barrier layer 0.050 mm thick. The nepafenac or other therapeutic agent is contained in the core EVA layer 1.3 mm thick which is covered by a 0.45 mm EVA film.

### Example 2: Additional Implant Device Fabrication

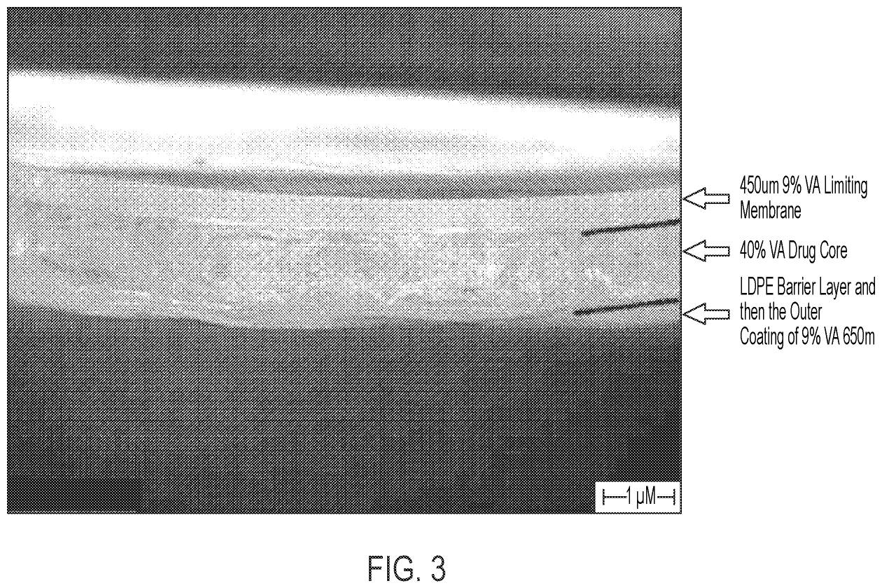

A preferred multi layer implant device was prepared by the procedures of Example 1 above. A photograph of the implant device cross-section is shown in FIG. 3. As depicted in FIG. 3, the implant device has an inner layer of vinyl acetate polymer with 5 weight percent of therapeutic agent. A 450 μm vinyl acetate (5%) layer adjoins one surface of the implant device layer and a low-density polyethylene (LDPE) layer abuts the opposing inner layer surface. An outer 450 μm vinyl acetate (5%) layer overcoats the LDPE layer.

### Example 3: Administration of a Multiple Layer Ocular Implant Device Containing Nepafenac in a Mouse Model of Dry AMD

Hydroquinone is known as an oxidant component of cigarette smoke. It has been found that mice treated with hydroquinone may be used as a model of dry AMD (Espinosa-Heidmann et al., Invest. Ophthal. Vis. Sci., 2006, 47-729). Aged male mice (>60 weeks, n=4) are fed a high fat diet (TD 88051; Harlan Teklad) supplemented with 0.8% hydroquinone for a minimum of 8 weeks. Alternatively, hydroquinone can be injected subconjunctivally for up 4 weeks as an alternate model. Other models of macular degeneration including the Y402H CFH transgenic under the control of the ApoE promoter, the Cc12−/− Cx3crl−/− mice, the SodI−/− mice, the OXY rats as well other animal models may also be used.

A multiple-layer ocular implant device as described in Example 1 above is administered to the treated mice. The implant devices are circular, 2.0 mm in diameter and 1 mm thick and contain nepafenac at doses of 3 mg and 5 mg.

It has been disclosed that placement of test implant devices in the sub-Tenon's space of rodents leads to episcleral clearance of the test substance (Chan, Pridgen and Csaky, 2010, Exp. Eye Res. 90,501). Therefore, surgical placement of the implants is performed by incising the conjunctiva and Tenon's fascia prior to placement of the devices in the sub-Tenon's space as far posteriorly as possible.

The mice are then monitored to determine the release of the drug over time by examining the eye using histology, electroretinography or changes in gene expression in the retinal pigment epithelium or photoreceptors. Confirmation of morphological changes in cells indicating the presence of the drug indicates the effectiveness of the implant device in transfer of nepafenac from the device to the surrounding tissues in the process of treating macular degeneration.

### Example 4: Administration Protocol

On Day 1, prior to test device administration, a subject's eye is be dilated with 1% tropicamide HCl and each subject receives buprenorphine (approximately 0.03 mg/kg SQ). The subject may be sedated for the injections using 20-50 mg/kg ketamine and 4-10 mg/kg xylazine IM, and the eyes can be aseptically prepared using topical 5% betadine solution, followed by rinsing with sterile eye wash. One drop of 0.5% proparacaine HCl then is applied. The superior conjunctiva is gently grasped with colibri forceps, and a 5-mm conjunctival incision is made 2-3 mm posterior and parallel to the limbus. Using Wescott scissors, the subtenon's space is opened and undermined superiorly. The implant is then be placed into the subtenon's space and the tenon and conjunctiva is closed with 8-0 or 9-0 nylon without tension. The implant of Example 1 can be used that include NACA for administration. The process can be repeated on the contralateral eye. Following the surgical procedure, digital photographs of implants may be taken and 1 drop of neomycin polymyxin B sulfates gramicidin ophthalmic solution or ofloxacin applied topically to the ocular surface.

### Example 5: Treatment of Patient with Macular Degeneration

A patient is diagnosed as suffering from age-related macular degeneration.

A four-layer ocular implant device is provided as described in Example 1 above (circular, 2.0 mm in diameter, 1.0 mm thick) and containing nepafenac at a dose of 3 mg. The implant device is placed behind the surface epithelium within the sub-Tenon's space. A lid speculum is placed and a conjunctival radial incision is made through the conjunctiva over the area where the implant device is to be placed. Wescott scissors are used to dissect posterior to Tenon's fascia and the implant device is inserted. The conjunctiva is reapproximated using a running 10-0 vicryl suture.

### EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

All cited sources, for example, references, publications, databases, database entries, and art cited herein, are incorporated into this application by reference, even if not expressly stated in the citation. In case of conflicting statements of a cited source and the instant application, the statement in the instant application shall control.

What is claimed is:

1. An ocular implant device comprising:
a first layer comprising a polymer;
a second layer 1) distinct from the first layer and 2) comprising one or more therapeutic agents, and
a third layer.

2. The device of claim 1 wherein the third layer is distinct from the first and second layers.

3. The device of claim 1 or 2 wherein the third layer comprises one or more rate-controlling agents.

4. The device of any one of claims 1 through 3 wherein the third layer is interposed between the first layer and second layer.

5. The device of any one of claims 1 through 4 wherein the cross-sectional thickness of the third layer is less than the cross-sectional thickness of the first layer.

6. The device of any one of claim 1 through S wherein the cross-sectional thickness of the third layer is less than the cross-sectional thickness of the second layer.

7. The device of any one of claims 1 through 6 wherein the third layer does not comprise a therapeutic agent.

8. The device of any one of claims 1 through 7 wherein the third layer comprises a polymer.

9. The device of claim 8 wherein the third layer comprises a polyimide.

10. The device of any one of claims 1 through 9 wherein the first layer does not contain a therapeutic agent.

11. The device of any one of claims 1 through 10 wherein the second layer comprises a Si-material.

12. The device of any one of claims 1 through 11 wherein the second layer comprises a polymer distinct from the first layer polymer.

13. The device of any one of claims 1 through 12 wherein the first layer extends circumferentially beyond the second layer such that the surface of the circumferential extension of the first hardened layer is capable of making contact with the sclera of the eye.

14. The device of any one of claims 1 through 13 wherein at least one surface of the second layer is capable of making contact with the sclera of a subject's eye.

15. The device of any one of claims 1 through 14 wherein the device comprises a fourth layer distinct from each of the first, second or third layer.

16. The device of any one of claims 1 through 15 wherein the device comprises a fourth layer distinct from an adjacent layer.

17. The device of claim 16 wherein the fourth layer is an outer layer of the device.

18. The device of claim 16 wherein the fourth layer is interposed between two other device layers.

19. The device of any one of claims 1 through 18 wherein the device comprises a fifth layer distinct from each of the first, second, third or fourth layer.

20. The device of any one of claims 1 through 26 wherein the device comprises a fifth layer distinct from an adjacent layer.

21. The device of claim 20 wherein the fifth layer is an outer layer of the device.

22. The device of claim 20 wherein the fifth layer is interposed between two other device layers.

23. The device of any one of claims 1 through 22 wherein the device comprises one or more of bromfenac; diclofenac; indomethacin; nepafenac; metformin; N-acetylcysteine amide (NAC amide); flurbiprofen; suprofen; and/or ketorolac, or a pharmaceutically acceptable salt thereof.

24. The device of any one of claims 1 through 22 wherein the device comprises one or more NSAID agents.

25. The device of any one of claims 1 through 24 wherein the device has a curved surface.

26. The device of any one of claims 1 through 25 wherein the device is circular or oval shape.

27. The device of any one of claims 1 through 26 wherein the device has a configuration that enhances anchoring upon implanting to a patient.

28. The device of any one of claims 1 through 27 wherein at least one layer of the device comprises a biodegradable or bioerodable material.

29. The device of any one of claims 1 through 27 wherein an at least one outer layer of the device comprises a biodegradable or bioerodable material.

30. The device of any one of claims 1 through 29 wherein at least one layer of the device comprises a polylactic-polyglycolic acid (PLGA) material.

31. The device of any one of claims 1 through 29 wherein at least one layer of the device comprises a poly(glycerol sebacate) (PGS) material.

32. The device of any one of claims 1 through 29 wherein at least one layer of the device comprises a poly (glycerol sebacate urethane) (PGSU) material.

33. The device of any one of claims 1 through 32 wherein at least one layer is substantially impermeable to diffusion of therapeutic agent.

34. The device of 33 wherein the at least one impermeable layer comprises one or more polymers of polyvinyl acetate, cross-linked polyvinyl alcohol, cross-linked polyvinyl butyrate, ethylene ethylacrylate co-polymer, polyethyl hexylacrylate, polyvinyl chloride, polyvinyl acetals, plasiticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumarate copolymer, silicone rubbers, medical grade polydimethylsiloxanes, ethylene-propylene rubber, silicone-carbonate copolymers, vinylidene chloride-vinyl chloride copolymer, vinyl chloride-acrylonitrile copolymer or vinylidene chloride-acrylonitride copolymer.

35. The device of any one of claims 1 through 34 wherein one or more layers are each about 1.5 mm or less thick.

36. The device of any one of claims 1 through 35 wherein one or more layers are each 1 mm or less thick.

37. The device of any one of claims 1 through 36 wherein one or more layers are each about 0.8 mm or less thick.

38. The device of any one of claims 1 through 37 wherein one or more layers are each about 0.5 mm or less thick.

39. The device of any one of claims 1 through 38 at least one layer comprises one or more of polyvinyl acetate, cross-linked polyvinyl alcohol, cross-linked polyvinyl butyrate, ethylene ethylacrylate co-polymer, polyethyl hexylacrylate, polyvinyl chloride, polyvinyl acetals, plasticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumarate copolymer, silicone rubbers, medical grade polydimethylsiloxanes, ethylene-propylene rubber, silicone-carbonate copolymers, vinylidene chloride-vinyl chloride copolymer, vinyl chloride-acrylonitrile copolymer or vinylidene chloride-acrylonitride copolymer.

40. The device of any one of claims 1 through 39 wherein at least one layer comprises an ophthalmic permeation agent that increases ocular permeability of the therapeutic agent into the eye.

41. The device of any one of claims 1 through 40 wherein a layer comprises a therapeutic agent admixed with a controlled release composition.

42. The device of any one of claims 1 through 41 wherein a layer comprises a controlled release composition that comprises one or more polymers.

43. The device of any one of claims 1 thought 42 wherein at least one device layer comprises one or more biodegradable polymers.

44. The device of any one of claims 1 thought 43 wherein at least one device layer comprises 1) one or more therapeutic agents and 2) one or more biodegradable polymers.

45. The device of claim 43 or 44 wherein the one or more biodegradable polymers degrade over extended time in a patient eye to thereby administer the one or more therapeutic agents to the patient.

46. The device of any one of claims 43 through 45 wherein the one or more biodegradable polymers comprise poly(lactic-co-glycolide), polylactic-polyglycolic acid block copolymers (PLGA), hydroxypropyl methyl cellulose, hydroxyl methyl cellulose, polyglycolide-polyvinyl alcohol, croscarmellose sodium, hydroxypropylcellulose, sodium carboxymethylcellulose, polyglycolic acid-polyvinyl alcohol copolymers (PGA/PVA), hydroxypropylmethylcellulose (HPMC), polycaprolactonepolyethylene glycol copolymers, poly(glycerol sebacate) (PGS) material and/or a poly (glycerol sebacate urethane) (PGSU) material.

47. A method for delivery of a therapeutic agent to a patient's eye comprising:
(a) providing an implant device of any one of claims 1 through 46 wherein the device comprises one or more therapeutic agents; and
(b) inserting the device into a patient's eye.

48. The method of claim 47 wherein the device is placed into the sub-Tenon's space and in contact with the sclera of the eye.

49. The method of claim 47 or 48 wherein the one or more therapeutic agents have a delivery duration of about two weeks to about 6 weeks.

50. The method of any one of claims 47 through 49 wherein the patient is suffering from macular degeneration.

51. The method of any one of claims 47 through 50 wherein the patient is suffering from age-related macular degeneration (AMD).

52. The method of any one of claims 47 through 51 wherein the device is placed in the posterior of the eye near the macula of the eye.

53. The method of any one of claims 47 through 52 wherein an applicator device is used to place the device into the sub-Tenon's space of the eye.

54. A kit comprising:
(a) an implant device of any one of claims 1 through 46; and
(b) instructions for use of the device to treat an eye disorder.

55. The kit of claim 54 further comprises an injector or other application device.
